(19) United States
(12) Patent Application Publication
(10) Pub. No.: US 2003/0208191 A1
(43) Pub. Date: Nov. 6, 2003

(54) Laser refractive surgical procedure method

(76) Inventors: Jeffrey T. Shimmel, Deltona, FL (US); Peter Austin, Orlando, FL (US); Alex C. Sacharoff, Oviedo, FL (US); Richard J. Potvin, Oviedo, FL (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/454,845

(22) Filed: Jun. 4, 2003

Related U.S. Application Data
(63) Continuation-in-part of application No. 10/235,302, filed on Sep. 5, 2002.
Application No. 10/235,302 is a continuation-in-part of application No. 10/114,834, filed on Apr. 3, 2002.

(51) Int. Cl. A61B 18/20

(52) U.S. Cl. 606/5

(57) ABSTRACT

A refractive surgical procedure method using microkeratome blade for use for the removal of the epithelial layer and underlying Basement Membrane while leaving a smooth and undisturbed Bowman's Membrane in preparation for a laser refractive surgical procedure. The blade is capable of cutting through the epithelial layer and Basement Membrane, but not capable of cutting through Bowman's Membrane. Prior to removing the epithelium, a solution suitable for reducing the adhesion of the epithelium is applied to the eye. Following surgery, a customized contact lens is placed on the eye. The contact lens may contain an agent to assist in the regeneration of the epithelium.

This application is a continuation-in-part of U.S. patent application Ser. No. 10/235,302, filed Sep. 5, 2002, currently co-pending, which is a continuation-in-part of U.S. patent application Ser. No. 10/114,834, filed Apr. 3, 2002, currently co-pending.

### BACKGROUND OF THE INVENTION

This invention relates generally to the field of refractive surgery and, more particularly, to microkeratomes used for performing laser refractive surgery. The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

The optical power of the eye is determined by the optical power of the cornea and the crystalline lens. In the normal, healthy eye, sharp images are formed on the retina (emmetropia). In many eyes, images are either formed in front of the retina because the eye is abnormally long (axial myopia) or the cornea is too steep (refractive myopia), or formed in back of the retina because the eye is abnormally short (axial hyperopia) or the cornea is too flat (refractive hyperopia). The cornea also may be asymmetric or toric, resulting in an uncompensated cylindrical refractive error referred to as corneal astigmatism. In addition, due to age-related reduction in lens accommodation, the eye may become presbyopic resulting in the need for a bifocal or multifocal correction device. In the past, myopia, hyperopia and corneal astigmatism generally have been corrected by spectacles or contact lenses, but there are several refractive surgical procedures that have been investigated and used since 1949. Jose Barraquer, M.D. investigated a procedure called keratomileusis that reshaped the cornea using a microkeratome and a cryolathe. This procedure was never widely accepted by surgeons. Another procedure that has gained widespread acceptance is radial and/or transverse incisional keratotomy (RK or AK, respectively). In the 1990s, the use of photoablative lasers to reshape the surface of the cornea (photorefractive keratectomy or PRK) or for mid-stromal photoablation (Laser-Assisted In Situ Keratomileusis or LASIK) have been approved by regulatory authorities in the U.S. and other countries. Recently, a new version of PRK called Laser Epithelial Keratomileusis (LASEK) has been developed wherein the epithelial layer is soaked in alcohol so as to release it from Bowman's Membrane and the epithelial layer is non-destructively rolled aside and the underlying stromal tissue is ablated in a manner similar to PRK. This procedure does not always allow for the smooth removal of the epithelial layer in a single sheet. In addition, alcohol is toxic to corneal tissue.

Accordingly, a need continues to exist for a device and method for the safe, consistent removal of the epithelial layer and Basement Membrane during a laser refractive surgical procedure.

### BRIEF SUMMARY OF THE INVENTION

The present invention improves upon the prior art by providing a refractive surgical procedure method using microkeratome blade for use for the removal of the epithelial layer and underlying Basement Membrane while leaving a smooth and undisturbed Bowman's Membrane in preparation for a laser refractive surgical procedure. The blade is capable of cutting through the epithelial layer and Basement Membrane, but not capable of cutting through Bowman's Membrane. Prior to removing the epithelium, a solution suitable for reducing the adhesion of the epithelium is applied to the eye. Following surgery, a customized contact lens is placed on the eye. The contact lens may contain an agent to assist in the regeneration of the epithelium.

Accordingly, one objective of the present invention is to provide a safe method for the removal of the epithelial layer and underlying Basement Membrane in preparation for a laser refractive surgical procedure.

Another objective of the present invention is to provide a method for the removal of the epithelial layer in preparation for a laser refractive surgical procedure without the use of toxic chemicals.

Another objective of the present invention is to provide a device that provides the safe and non-toxic method for the removal of the epithelial layer and underlying Basement Membrane in preparation for a laser refractive surgical procedure.

Another objective of the present invention is to provide a microkeratome blade that provides the safe and method for the removal of the epithelial layer and underlying Basement Membrane in preparation for a laser refractive surgical procedure.

These and other advantages and objectives of the present invention will become apparent from the detailed description and claims that follow.

### BRIEF DESCRIPTION OF THE DRAWING

FIG. 1 is a schematic representation of a microkeratome that may be used with the invention of the present method.

FIG. 2 is an enlarged partial side view of the microkeratome blade of the present invention.

FIG. 3 is a partial cross-sectional view of a human cornea.

FIG. 4 is a perspective view of second embodiment of a head that may be used on the microkeratome illustrated in FIG. 1.

FIG. 5 is a schematic representation of an eye having a therapeutic contact lens constructed according to the teaching of the present invention.

FIG. 6 is a top plan view of an annular well that may be used with the presnet invention.

### DETAILED DESCRIPTION OF THE INVENTION

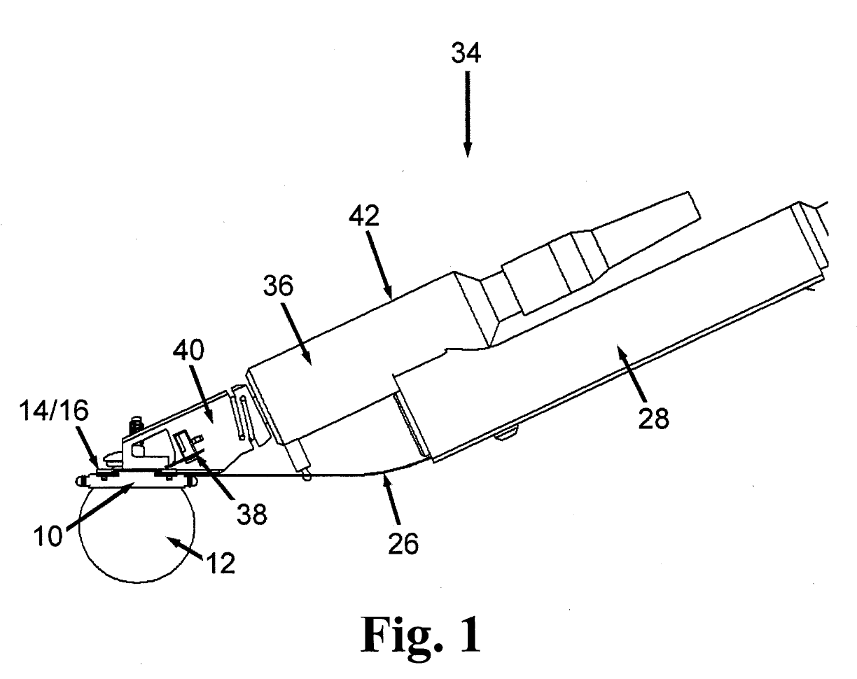

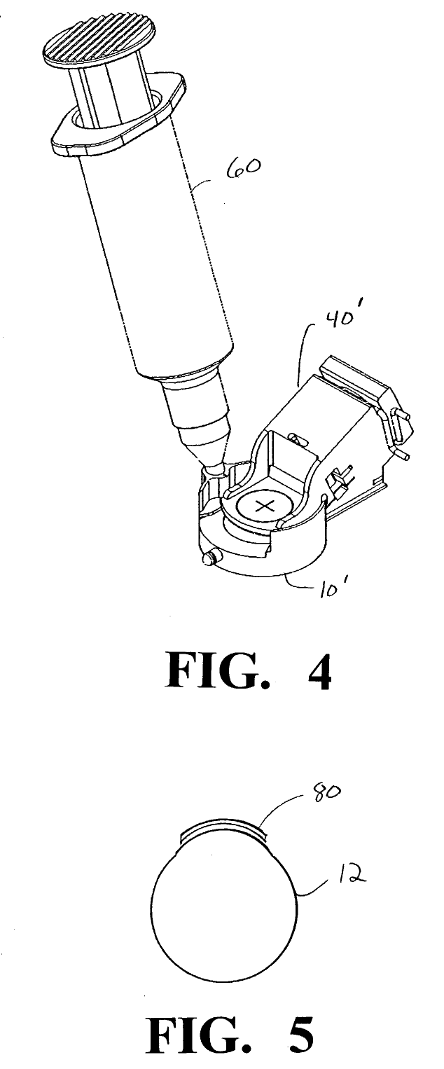

As best seen in FIG. 1, one microkeratome 34 that may be used with the method of the present invention generally includes suction ring 10 sized and shaped so as to affix to eye 12. Ring 10 includes guides 14/16 opposite eye 12 that guide cutting head 40 across ring 10. Ring 10 is connected through translation member 26 to stepper motor 28 for providing linear movement of cutting head 40 across ring 10. Cutting head 40 contains blade 38 that is eccentrically connected to motor 36 contained within housing 42 of microkeratome 34. Microkeratome 34 is well known in the art (see for example U.S. Pat. No. 6,071,293 (Krumeich), the entire contents of which being incorporated herein by reference). Alternatively, as shown in FIG. 4, head 40′ may contain ring 10′ to which syringe 60 is fluidly connected. Syringe 60 is used to draw a vacuum in ring 10′ and thereby affix ring 10′ to eye 12. The use of syringe 60 eliminates the need for any outside source of vacuum to ring 10 or 10′. Head 40′, including ring 10′ and syringe 60 may be made to be disposable.

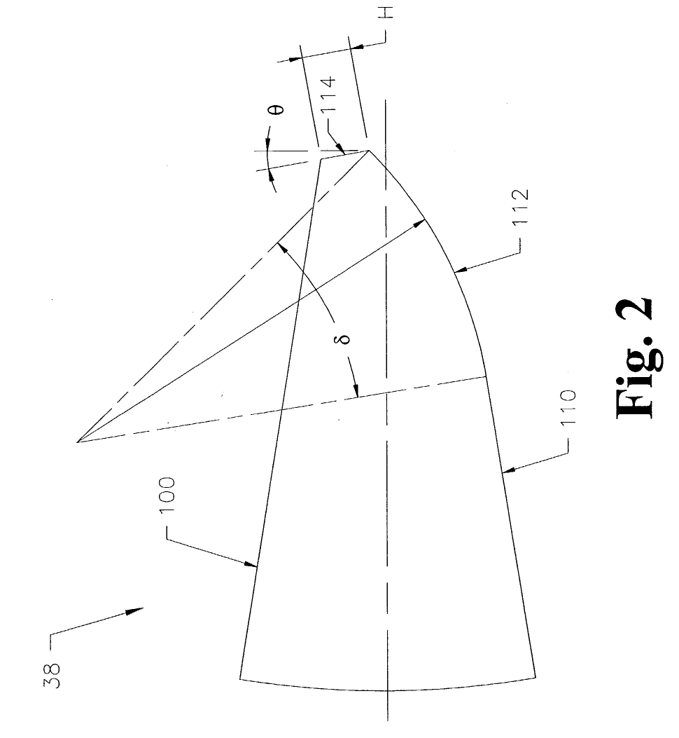

As best seen in FIG. 2, blade 38 generally includes relatively flat side 100, tapered side 110 containing rounded section 112 and blunt tip 114 connecting flat side 100 and rounded section 112. Rounded section 112 generally has a radius of between about 0.025 millimeters and 0.200 millimeters and is rounded through of angle δ of between approximately 5 degrees and 60 degrees. Blunt tip 114 generally has a length L of between approximately 0.001 millimeters and 0.050 millimeters, with between about 0.005 millimeters and 0.025 millimeters being preferred, and is ground at an offset angle Θ relative to rounded portion 112 at between approximately between 0 degrees and 60 degrees, with between approximately between 0 degrees and 20 degrees being preferred. Preferably, rounded section 112 and blunt tip 114 have a textured surface finish. Blade 38 may be made of any suitable material, such as 400 Series stainless steel and may be made using conventional surgical blade manufacturing techniques well known in the art.

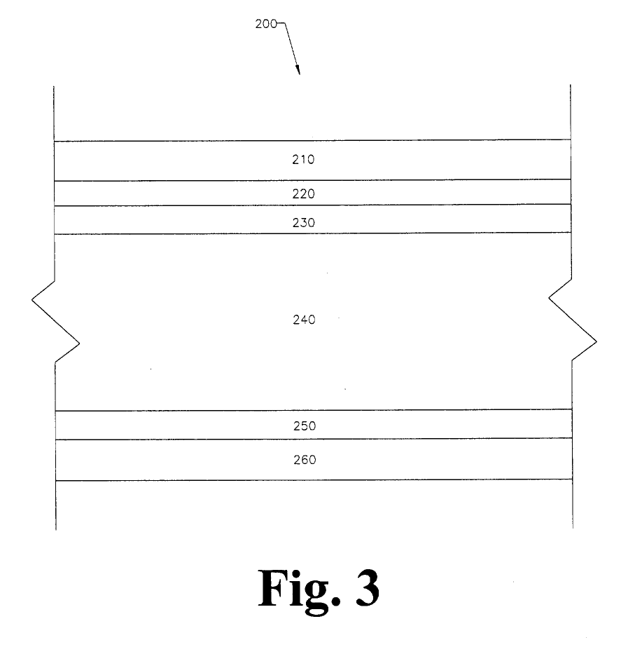

As best seen in FIG. 3, human cornea 200 has several layers. The outermost layer is epithelium 210, followed by Basement Membrane 220, Bowman's Membrane 230, substantia propria or stroma 240, Descemet's Membrane 250 and endothelium 260. The method of the present invention involves the use of microkeratome 34 having blade 38 to remove epithelium 210 and Basement Membrane 220 while leaving Bowman's Membrane 230 relatively intact. The method of the present invention uses microkeratome 34 in a conventional manner well known to those skilled in the art. Preferably, the intraocular pressure of the eye undergoing the surgical procedure is briefly raised to around 80 mm Hg or greater. The oscillation frequency of blade 38 preferably is approximately between 5,000 revolutions/minute and 20,000 revolutions/minute, with approximately between 8,000 revolutions/minute and 14,000 revolutions/minute being most preferred. The speed of blade 38 as it traverses cornea 200 preferably is approximately between 1.0 millimeter/second and 2.0 millimeters/second, with approximately 1.5 millimeters/second being most preferred. As blade 38 approaches cornea 200, blunt tip 114 penetrates epithelium 210 and Basement Membrane 220, but is insufficiently sharp to penetrate Bowman's Membrane 230. As a result, blunt tip 114 and rounded portion 112 scrape along the surface of Bowman's Membrane 230, separating epithelium 210 and Basement Membrane 220 from Bowman's Membrane 230 without damaging Bowman's Membrane 230. Following such separation, Bowman's Membrane 230 and stroma 240 are irradiated as in a conventional laser refractive surgical procedure, see for example, U.S. Pat. Nos. 4,784,135 (Blum, et al.) and 4,903,695 C1 (Warner, et al.), the entire contents of which being incorporated herein by reference.

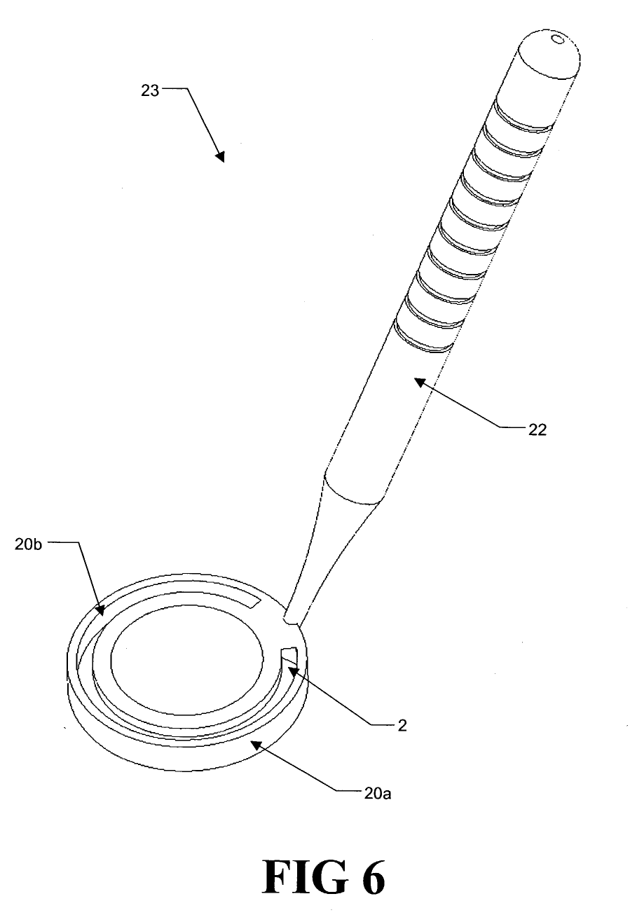

To facilitate the removal of epithelium 210 and Bowman's Membrane 230, a suitable pharmaceutical agent may be placed on eye 12 prior to the introduction of ring 10 or 10′, such as a balanced salt solution or ringers solution, both of which have a tendency to reduce the adhesion of epithelium 210 to Basement Membrane 220 and are not toxic to eye 12. Other adhesion reducing agents, such as alcohol, may also be used, although non-toxic agents are preferred. The agent may be applied using a cylindrical or annular well, the former known in the art. The latter will restrict application of the agent to the periphery of the area of the cornea to be removed. One suitable annular well 23 is illustrated in FIG. 6. Well 23 has a ring 20 having an outer wall 20a and an inner wall 20b and channel 21 intermediate walls 20a and 20b for holding an agent on the eye. Well 23 may be held in position using handle 22. Following the laser refractive surgical procedure, a pharmaceutical agent suitable for invigorating the health and viability of epithelium 210, such as lactated ringers solution or a solution containing 5% dextrose (Dextran), may be applied to eye 12. The retention of this agent may be enhanced by the placement of high water content contact lens 80 on eye 12. Preferably, such contact lens 80 is custom designed and sized to fit tightly eye 12, allowing for any tissue removed by the laser refractive procedure, as seen in FIG. 5. Such a contact lens design generally shall be constructed using one or more of several measurements taken of eye 12, such as a wavefront analysis, manifest refraction (sphere, cylinder, axis), cycloplegic refraction, anterior and posterior corneal surface shape, location of the anterior and posterior surfaces of the natural lens, axial length and curvature of the retinal pole.

This description is given for purposes of illustration and explanation. It will be apparent to those skilled in the relevant art that changes and modifications may be made to the invention described above without departing from its scope or spirit.

We claim:

1. A method of performing a laser refractive surgical procedure, comprising the steps of:
a) applying a first pharmaceutical agent to an eye, the first pharmaceutical agent suitable for reducing an adhesion of an epithelium to a Basement Membrane;
b) raising an intraocular pressure of the eye having a cornea to around 80 mm Hg or higher;
c) contacting the cornea with a blade;
d) advancing the blade across the cornea so that the blade penetrates the epithelium so as to expose but not penetrate the Bowman's Membrane of the cornea;
e) irradiating the Bowman's Membrane and underlying stromal tissue with ablative laser radiation to effect a refractive change in the cornea; and
f) applying a second pharmaceutical agent to the eye, the second pharmaceutical agent suitable for invigorating the health and viability of the epithelium.

2. The method of claim 1 wherein the blade contains a blunt tip.

3. The method of claim 2 wherein the blade has a flat section and a rounded section and the blunt tip separates the flat section from the rounded section.

4. The method of claim 1 wherein the blade is oscillated at an oscillation frequency of approximately between 5,000 revolutions/minute and approximately 20,000 revolutions/minute.

5. The method of claim 4 wherein the oscillation frequency is approximately between 8,000 revolutions/minute and 14,000 revolutions/minute.

6. The method of claim 1 wherein the blade is advanced across the cornea at a speed of approximately between 1.0 millimeter/second and 2.0 millimeters/second.

7. The method of claim 6 wherein the blade is advanced across the cornea at a speed of approximately 1.5 millimeters/second.

8. A method of performing a laser refractive surgical procedure, comprising the steps of:
a) applying a first pharmaceutical agent to an eye, the first pharmaceutical agent suitable for reducing an adhesion of an epithelium to a Basement Membrane;
b) raising an intraocular pressure of a eye having a cornea to around 80 mm Hg or higher;
c) contacting the with a blade, the blade oscillated at an oscillation frequency of approximately between 5,000 revolutions/minute and 20,000 revolutions/minute, the intraocular pressure of the eye being raised to 80 mm Hg or higher;
d) advancing the blade across the cornea at a speed of approximately between 1.0 millimeter/second and 2.0 millimeters/second so that the blade penetrates the epithelium so as to expose but not penetrate a Bowman's Membrane of the cornea;
e) irradiating the Bowman's Membrane and underlying stromal tissue with ablative laser radiation to effect a refractive change in the cornea; and
f) applying a second pharmaceutical agent to the eye, the second pharmaceutical agent suitable for invigorating the health and viability of the epithelium.

9. The method of claim 8 wherein the blade contains a blunt tip.

10. The method of claim 9 wherein the blade has a flat section and a rounded section and the blunt tip separates the flat section from the rounded section.

11. The method of claim 8 wherein the oscillation frequency is approximately between 8,000 revolutions/minute and 14,000 revolutions/minute.

12. The method of claim 8 wherein the blade is advanced across the cornea at a speed of approximately 1.5 millimeters/second.

13. A method of performing a laser refractive surgical procedure, comprising the steps of:
a) measuring an eye, such measurements including one of more of a wavefront analysis, a manifest refraction, a cycloplegic refraction, an anterior and posterior corneal surface shape, a location of the anterior and posterior surfaces of the natural lens, an axial length and a curvature of the retinal pole
b) applying a first pharmaceutical agent to an eye, the first pharmaceutical agent suitable for reducing an adhesion of an epithelium to a Basement Membrane;
c) raising an intraocular pressure of the eye having a cornea to around 80 mm Hg or higher;
d) contacting the cornea with a blade;
e) advancing the blade across the cornea so that the blade penetrates the epithelium so as to expose but not penetrate the Bowman's Membrane of the cornea;
f) irradiating the Bowman's Membrane and underlying stromal tissue with ablative laser radiation to effect a refractive change in the cornea; and
g) applying a second pharmaceutical agent to the eye, the second pharmaceutical agent suitable for invigorating the health and viability of the epithelium.

14. The method of claim 13 wherein the blade contains a blunt tip.

15. The method of claim 14 wherein the blade has a flat section and a rounded section and the blunt tip separates the flat section from the rounded section.

16. The method of claim 13 wherein the blade is oscillated at an oscillation frequency of approximately between 5,000 revolutions/minute and approximately 20,000 revolutions/minute.

17. The method of claim 16 wherein the oscillation frequency is approximately between 8,000 revolutions/minute and 14,000 revolutions/minute.

18. The method of claim 13 wherein the blade is advanced across the cornea at a speed of approximately between 1.0 millimeter/second and 2.0 millimeters/second.

19. The method of claim 18 wherein the blade is advanced across the cornea at a speed of approximately 1.5 millimeters/second.

20. The method of claim 13 further comprising the step of placing a contact lens on the eye to assist in the retention of the second pharmaceutical agent.

21. The method of claim 20 wherein the contact lens is designed using the measurements taken of the eye.
